(19) United States
(12) Patent Application Publication
(10) Pub. No.: US 2026/0157692 A1
(43) Pub. Date: Jun. 11, 2026

(54) METHOD AND APPARATUS FOR EVALUATING PAIN IN PAIN DIAGNOSIS SYSTEM

(71) Applicant: Seoul National University Hospital, Seoul (KR)

(72) Inventors: Insun PARK, Seoul (KR); Jae Hyon PARK, Seoul (KR)

(21) Appl. No.: 19/333,911

(22) Filed: Sep. 19, 2025

(30) Foreign Application Priority Data
Sep. 20, 2024 (KR) 10-2024-0127731

(51) Int. Cl. A61B 5/00

(52) U.S. Cl. CPC A61B 5/4824; A61B 5/0077; A61B 5/7264; A61B 5/742; A61B 5/746

(57) ABSTRACT

There is provided a method for evaluating pain in a pain diagnosis system, the method comprising: determining a target BIS (bispectral index) range that includes a target BIS measured from a target patient; and evaluating the pain of the target patient by using at least one of a first pain score related to an ANI (analgesia nociception index) and a second pain score related to an AU (action unit) representing facial expressions, based on the target BIS range.

### CROSS REFERENCE TO RELATED APPLICATION

The present application claims priority to Korean Patent Application No. 10-2024-0127731, filed Sep. 20, 2024, the entire contents of which are incorporated here for all purposes by this reference.

### TECHNICAL FIELD

An embodiment relates to a method and an apparatus for evaluating pain in a pain diagnosis system.

### BACKGROUND

Pain is a subjective experience that a patient complains of, so it is difficult to evaluate accurately. Evaluation of subjective pain is performed via the patient-reported numeric rating scale (NRS), but this is a method that requires a patient to personally state their pain intensity on a scale of 0 to 10.

Here, although guidelines for the criteria for determining pain intensity between 0 and 10 are provided, the criteria may be considered to be different for each patient, and there is a limitation in that pain evaluation for patients with communication difficulties cannot be appropriately performed.

Various attempts have been made to objectively evaluate pain, but conventional pain evaluation methods have a problem in that the accuracy of pain evaluation varies depending on the type of patient (e.g., chronic pain patients and acute pain patients, unconscious patients and conscious patients).

Accordingly, there is a need to develop a technology for objectively evaluating a target patient's pain regardless of the patient's type by differently applying an appropriate pain evaluation method based on the patient's BIS.

### SUMMARY

An object of an embodiment is to objectively assess pain regardless of a patient's type by distinguishing between a case where the patient is conscious and a case where the patient is unconscious based on the patient's BIS value, and by differently applying an appropriate pain evaluation method corresponding to a case where the patient is conscious and a case where the patient is unconscious.

Furthermore, an object of an embodiment is to provide a one-stop service for pain management that leads to automated pain evaluation, providing a notification to medical staff, and administering medication after a patient's surgery.

In accordance with an aspect of the present disclosure, there is provided a method for evaluating pain in a pain diagnosis system, the method comprising: determining a target BIS (bispectral index) range that includes a target BIS measured from a target patient; and evaluating the pain of the target patient by using at least one of a first pain score related to an ANI (analgesia nociception index) and a second pain score related to an AU (action unit) representing facial expressions, based on the target BIS range.

The target BIS range comprises: a first range where the BIS is 60 or less; a second range where the BIS is over 80; and a third range where the BIS is over 60 and up to 80, and the determining the target BIS range comprises: determining a range among the first range, the second range, and the third range that includes the target BIS.

When the target BIS is included in the first range, the evaluating the pain of the target patient comprises: evaluating the pain of the target patient using the first pain score.

When the target BIS is included in the second range, the evaluating the pain of the target patient comprises: extracting features for the AU from a facial image of the target patient; determining the second pain score by inputting the features for the AU into a pre-trained artificial intelligence model; and evaluating the pain of the target patient using the second pain score.

The artificial intelligence model is trained to determine the second pain score based on a training dataset comprising a facial image of a patient and an NRS (numeric rating scale) label corresponding to the pain of the patient.

When the target BIS is included in the third range, the evaluating the pain of the target patient comprises: evaluating the pain of the target patient using the first pain score and the second pain score.

The evaluating the pain of the target patient comprises: determining a third pain score by calculating a weighted sum of the first pain score and the second pain score, based on a weight determined from the target BIS; and evaluating the pain of the target patient using the third pain score.

The method further comprising: displaying at least one pain score among the first pain score, the second pain score, and a third pain score, based on monitoring of the target BIS.

The displaying the at least one pain score comprises: providing an alarm to medical staff when at least one of the first pain score, the second pain score, and the third pain score exceeds a preset threshold value.

In accordance with an aspect of the present disclosure, there is provided an apparatus for evaluating pain in a pain diagnosis system, the apparatus comprising: a memory storing a pain evaluation program including one or more instructions; and a processor that loads the pain evaluation program from the memory and executes the pain evaluation program, wherein the one or more instructions, when executed by the processor, cause the processor to: determine a target BIS (bispectral index) range that includes a target BIS measured from a target patient, and evaluate the pain of the target patient by using at least one of a first pain score related to an ANI (analgesia nociception index) and a second pain score related to an AU (action unit) representing facial expressions, based on the target BIS range.

The target BIS range comprises: a first range where the BIS is 60 or less; a second range where the BIS is over 80; and a third range where the BIS is over 60 and up to 80, and the one or more instructions, when executed by the processor, cause the processor to determine a range among the first range, the second range, and the third range that includes the target BIS.

When the target BIS is included in the first range, the one or more instructions, when executed by the processor, cause the processor to evaluate the pain of the target patient using the first pain score.

When the target BIS is included in the second range, the one or more instructions, when executed by the processor, cause the processor to: extract features for the AU from a facial image of the target patient, determine the second pain score by inputting the features for the AU into a pre-trained artificial intelligence model, and evaluates the pain of the target patient using the second pain score.

The artificial intelligence model is trained to determine the second pain score based on a training dataset comprising a facial image of a patient and an NRS (numeric rating scale) label corresponding to the pain of the patient.

When the target BIS is included in the third range, the one or more instructions, when executed by the processor, cause the processor to evaluate the pain of the target patient using the first pain score and the second pain score.

The one or more instructions, when executed by the processor, cause the processor to: determine a third pain score by calculating a weighted sum of the first pain score and the second pain score, based on a weight determined from the target BIS, and evaluates the pain of the target patient using the third pain score.

In accordance with an aspect of the present disclosure, there is provided a pain diagnosis system, comprising: a BIS measurement unit that measures a target BIS of a target patient; an ANI measurement unit that measures an ANI of the target patient; an imaging unit that acquires a facial image of the target patient; a pain evaluation apparatus that determines a target BIS range that includes the target BIS, and evaluates the pain of the target patient using a pain score determined based on the target BIS range; a display unit that displays the target BIS, the ANI, and the pain score; and a monitoring unit that, through monitoring of the target BIS, provides an alarm to medical staff when the pain score exceeds a preset threshold value.

In accordance with an aspect of the present disclosure, there is provided a computer-readable recording medium storing a computer program, wherein the computer program, when executed by a processor, causes the processor to: determine a target BIS (bispectral index) range that includes a target BIS measured from a target patient; and evaluate the pain of the target patient by using at least one of a first pain score related to an ANI (analgesia nociception index) and a second pain score related to an AU (action unit) representing facial expressions, based on the target BIS range.

According to an embodiment, by differently applying an appropriate pain evaluation method in consideration of the patient's condition, pain may be objectively and accurately assessed regardless of the patient's type.

Furthermore, according to an embodiment, pain management for a patient may be performed promptly and appropriately by providing a one-stop service for pain management through a pain diagnosis system, which leads to automated pain evaluation, providing a notification to medical staff, and administering medication after the patient's surgery.

Furthermore, according to an embodiment, it is possible to reduce the continuous fatigue of medical professionals and medical costs for pain evaluation.

### BRIEF DESCRIPTION OF THE DRAWINGS

FIG. 1 is a diagram illustrating a pain diagnosis system according to an embodiment.

FIG. 2 is a block diagram illustrating a pain evaluation apparatus according to an embodiment.

FIG. 3 is a block diagram conceptually illustrating functions of a pain evaluation program according to an embodiment.

FIG. 4 is a flowchart illustrating a pain evaluation method according to an embodiment.

### DETAILED DESCRIPTION

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

Terms used in the present specification will be briefly described, and the present disclosure will be described in detail.

In terms used in the present disclosure, general terms currently as widely used as possible while considering functions in the present disclosure are used. However, the terms may vary according to the intention or precedent of a technician working in the field, the emergence of new technologies, and the like. In addition, in certain cases, there are terms arbitrarily selected by the applicant, and in this case, the meaning of the terms will be described in detail in the description of the corresponding invention. Therefore, the terms used in the present disclosure should be defined based on the meaning of the terms and the overall contents of the present disclosure, not just the name of the terms.

When it is described that a part in the overall specification “includes” a certain component, this means that other components may be further included instead of excluding other components unless specifically stated to the contrary.

In addition, a term such as a “unit” or a “portion” used in the specification means a software component or a hardware component such as FPGA or ASIC, and the “unit” or the “portion” performs a certain role. However, the “unit” or the “portion” is not limited to software or hardware. The “portion” or the “unit” may be configured to be in an addressable storage medium, or may be configured to reproduce one or more processors. Thus, as an example, the “unit” or the “portion” includes components (such as software components, object-oriented software components, class components, and task components), processes, functions, properties, procedures, subroutines, segments of program code, drivers, firmware, microcode, circuits, data, database, data structures, tables, arrays, and variables. The functions provided in the components and “unit” may be combined into a smaller number of components and “units” or may be further divided into additional components and “units”.

Hereinafter, the embodiment of the present disclosure will be described in detail with reference to the accompanying drawings so that those of ordinary skill in the art may easily implement the present disclosure. In the drawings, portions not related to the description are omitted in order to clearly describe the present disclosure.

Hereinafter, embodiments of the present disclosure will be described in detail with reference to the attached drawings.

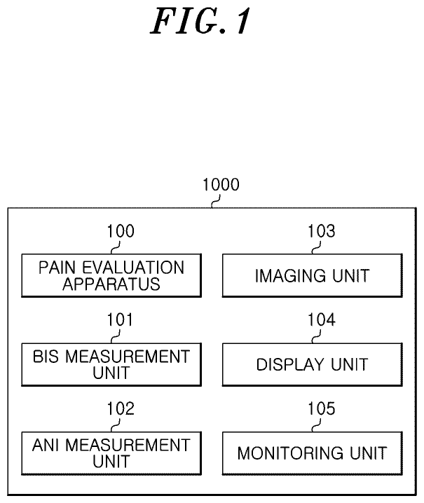

FIG. 1 is a diagram illustrating a pain diagnosis system according to an embodiment.

Referring to FIG. 1, a pain diagnosis system 1000 according to an embodiment may include a pain evaluation apparatus 100, a BIS measurement unit 101, an ANI measurement unit 102, an imaging unit 103, a display unit 104, and a monitoring unit 105.

The pain evaluation apparatus 100 may determine a target BIS range that includes a target BIS, and evaluate pain of a target patient by using a pain score determined based on the target BIS range.

The BIS measurement unit 101 may measure a target BIS of a target patient by using a predetermined sensor. Specifically, the BIS measurement unit 101 may quantify a degree of sedation of the patient through measurement of an electroencephalogram index.

Specifically, the ANI measurement unit 102 may quantify a patient's degree of pain by measuring an ECG or EKG waveform. This measurement is based on the concepts that the RR interval of an electrocardiogram changes due to the autonomic nervous system, and that the RR interval changes with respiration according to changes in the parasympathetic nerve.

The imaging unit 103 may acquire a facial image of the target patient by using at least one camera.

The display unit 104 may display the dynamically changing target BIS, ANI, and pain score.

The monitoring unit 105, through monitoring of the target BIS, may provide an alarm to medical staff when the pain score exceeds a preset threshold value. Here, the preset threshold value may refer to a pain score at which medication administration for the target patient is required, and may be determined by medical staff.

As such, it is possible to provide a one-stop service for pain management through the pain diagnosis system, leading to automated pain evaluation after a patient's surgery, providing a notification to medical staff, and administering medication.

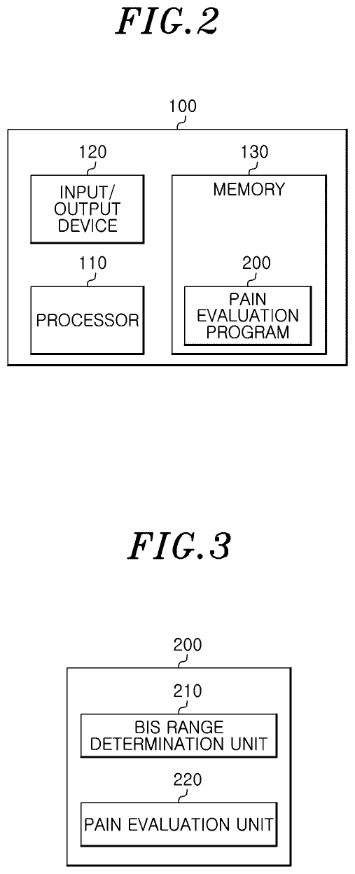

FIG. 2 is a block diagram illustrating a pain evaluation apparatus according to an embodiment.

Referring to FIG. 2, the pain evaluation apparatus 100 may include a processor 110, an input/output device 120, and a memory 130.

The processor 110 may generally control the operation of the pain evaluation apparatus 100.

The processor 110 may receive a facial image of a target patient by using an input/output device 120. Here, the facial image may refer to an image in which the front or side of the target patient is captured through a camera.

Furthermore, the processor 110 may receive a BIS (bispectral index) value of a target patient by using an input/output device 120.

Furthermore, the processor 110 may receive an ANI (analgesia nociception index) index of a target patient by using an input/output device 120.

In this specification, it has been described that at least one of the target patient's facial image, BIS value, and ANI index is input through the input/output device 120, but embodiments are not limited thereto. That is, according to an embodiment, the pain evaluation apparatus 100 may include a transceiver (not shown), and the pain evaluation apparatus 100 may receive at least one of the target patient's facial image, BIS value, and ANI index by using the transceiver (not shown), and at least one of the target patient's facial image, BIS value, and ANI index may be generated within the pain evaluation apparatus 100.

The processor 110 may determine a target BIS range that includes a target BIS measured from a target patient, and evaluate the pain of the target patient by using at least one of a first pain score related to ANI and a second pain score related to an AU representing facial expressions, based on the target BIS range.

The input/output device 120 may include one or more input devices and/or one or more output devices. For example, the input devices may include a microphone, a keyboard, a mouse, a touch screen, etc., and the output devices may include a display, a speaker, etc.

The memory 130 may store a pain evaluation program 200 and information necessary for execution of the pain evaluation program 200.

In this specification, the pain evaluation program 200 may refer to software including instructions for evaluating a target patient's pain using at least one of a first pain score related to ANI and a second pain score related to an AU representing facial expressions, based on a target BIS range.

The processor 110 may load the pain evaluation program 200 and information necessary for executing the pain evaluation program 200 from the memory 130 to execute the pain evaluation program 200.

The processor 110 may determine the first pain score related to ANI by executing the pain evaluation program 200.

Furthermore, the processor 110 may determine the second pain score by receiving as input features for at least one AU representing facial expressions, by executing the pain evaluation program 200. Meanwhile, for more details regarding the determination of the second pain score by inputting features for at least one AU according to an embodiment, reference is made to Korean Patent Application No. 10-2024-0127702, filed by the present applicant, the contents of which are incorporated herein by reference in their entirety.

The functions and/or operations of the pain evaluation program 200 will be described in detail with reference to FIG. 3.

FIG. 3 is a block diagram conceptually illustrating functions of a pain evaluation program according to an embodiment.

Referring to FIG. 3, the pain evaluation program 200 may include a BIS range determination unit 210 and a pain evaluation unit 220.

The BIS range determination unit 210 and the pain evaluation unit 220 shown in FIG. 3 are conceptual divisions for ease of explanation and are not intended to be limiting.

First, the BIS range determination unit 210 may determine the target BIS range that includes the target BIS measured from the target patient.

Here, the target BIS range is distinguished based on the depth of anesthesia, and may include a first range where the BIS is 60 or less, a second range where the BIS is over 80, and a third range where the BIS is over 60 and up to 80.

Specifically, the first range, where the BIS is 60 or less, may refer to a range corresponding to a state where the target patient is unconscious, such as under general anesthesia. Furthermore, the second range, where the BIS is over 80, may refer to a range corresponding to a state where the target patient is conscious and awake. Furthermore, the third range, where the BIS is over 60 and up to 80, may refer to a range corresponding to a state where the target patient is conscious but sedated.

Specifically, the BIS range determination unit 210 may determine a range among the first range, the second range, and the third range that includes the target BIS.

Meanwhile, the criteria for the BIS values that determine the first to third ranges according to an embodiment are merely an example, and may be variously modified within a scope that may achieve the object of an embodiment.

Next, the pain evaluation unit 220 may evaluate the pain of the target patient by using at least one of a first pain score related to ANI and a second pain score related to an AU representing facial expressions, based on the target BIS range.

Here, the first pain score related to ANI may correspond to an ANI value related to the patient's pain, and may refer to a discrete pain score having a range of 0 to 100 points.

Furthermore, according to an embodiment, the second pain score may correspond to the NRS (numeric rating scale) related to the patient's pain, and may refer to a discrete pain score having a range of 0 to 10 points.

Specifically, when the target BIS is included in the first range, the pain evaluation unit 220 may evaluate the pain of the target patient using the first pain score.

When the target patient is in a state of deep anesthesia and is unconscious, the pain of the target patient may be accurately evaluated using the first pain score related to ANI. However, since the ANI value is measured using respiratory fluctuations, it is difficult to accurately evaluate pain when the target patient is in an awake state.

Meanwhile, when the target BIS is included in the second range, the pain evaluation unit 220 may evaluate the pain of the target patient using the second pain score.

More specifically, the pain evaluation unit 220 may extract features related to an AU from a facial image of the target patient. Here, an AU is a unit of facial movement determined through facial components (e.g., eyebrows, eyes, nose, mouth, etc.), and may be included in an AU set (e.g., AU1 to AU64) of the FACS (facial action coding system), which is a method for describing facial expressions through the movement of muscles in the human face based on anatomy.

Furthermore, the features for the AU may be extracted through a predetermined algorithm (e.g., the OpenFace algorithm), and may refer to a binarized value (e.g., 0 or 1) that determines whether the facial image corresponds to an AU related to the upper facial area, lower facial area, facial orientation, or gaze, or a value related to the intensity corresponding to the AU.

Furthermore, the pain evaluation unit 220 may determine the second pain score by inputting the features for the AU into a pre-trained artificial intelligence model.

Here, the artificial intelligence model according to an embodiment may be one that is trained to determine a pain score based on a training dataset including a facial image of a patient and an NRS label corresponding to the patient's pain.

The NRS label according to an embodiment may refer to a multi-label that classifies the patient's facial image from 0 to 10 based on a medical professional's evaluation, or a binarized label that classifies the patient's facial image as 0 (i.e., when NRS is less than 7) or 1 (i.e., when NRS is 7 or more) based on the medical professional's evaluation.

More specifically, the artificial intelligence model according to an embodiment may be trained to determine a pain score based on a weight assigned to an AU associated with the shape of the patient's lips.

When the target patient is an acute pain patient or a post-operative pain patient and is in an awake state, the pain of the target patient may be accurately evaluated using the second pain score determined through the artificial intelligence model trained with an emphasis on the AU associated with the shape of the lips.

Meanwhile, when the target BIS is included in the third range, the pain evaluation unit 220 may evaluate the pain of the target patient using the first pain score and the second pain score.

More specifically, the pain evaluation unit 220 may evaluate the pain of the target patient using a third pain score determined based on the first pain score and the second pain score.

Here, the pain evaluation unit 220 may determine the third pain score by calculating a weighted sum of the first pain score and the second pain score, based on a weight determined from the target BIS.

For example, when the target BIS is 70, the pain evaluation unit 220 may determine a first weight for the first pain score and a second weight for the second pain score as 0.5 each, and may determine the third pain score by calculating a weighted sum of the first pain score and the second pain score based on the first weight and the second weight.

Meanwhile, in determining the third pain score, the pain evaluation unit 220 may perform preprocessing or normalization for correcting a scale of the first pain score and the second pain score.

As such, by differently applying an appropriate pain evaluation method in consideration of the patient's condition, a unique effect may be achieved in which pain may be objectively and accurately assessed regardless of the patient's type.

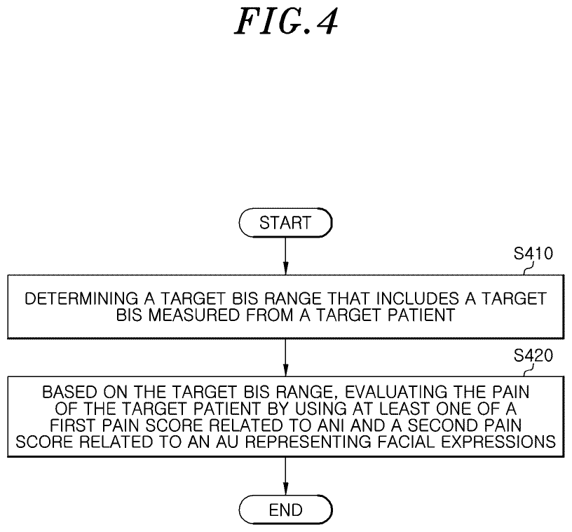

FIG. 4 is a flowchart illustrating a pain evaluation method according to an embodiment.

Referring to FIG. 1, FIG. 3, and FIG. 4, the BIS range determination unit 210 may determine a target BIS range that includes a target BIS measured from a target patient (S410). Here, the BIS range determination unit 210 may determine a range that includes the target BIS among a first range to a third range determined according to the BIS value.

Next, the pain evaluation unit 220 may evaluate the pain of the target patient by using at least one of a first pain score related to ANI and a second pain score related to an AU representing facial expressions, based on the target BIS range (S420).

Here, when the target BIS is included in the first range, the pain evaluation unit 220 may evaluate the pain of the target patient using the first pain score.

Furthermore, when the target BIS is included in the second range, the pain evaluation unit 220 may evaluate the pain of the target patient using the second pain score.

Furthermore, when the target BIS is included in the third range, the pain evaluation unit 220 may evaluate the pain of the target patient using a third pain score determined by calculating a weighted sum of the first pain score and the second pain score.

Meanwhile, the display unit 104 may display at least one of the first pain score, the second pain score, and the third pain score, based on monitoring of the target BIS and ANI.

Furthermore, the monitoring unit 105 may provide an alarm to medical staff when at least one of the first pain score, the second pain score, and the third pain score exceeds a preset threshold value.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

What is claimed is:

1. A method for evaluating pain in a pain diagnosis system, the method comprising:
determining a target BIS (bispectral index) range that includes a target BIS measured from a target patient; and
evaluating the pain of the target patient by using at least one of a first pain score related to an ANI (analgesia nociception index) and a second pain score related to an AU (action unit) representing facial expressions, based on the target BIS range.

2. The method of claim 1, wherein
the target BIS range comprises:
a first range where the BIS is 60 or less;
a second range where the BIS is over 80; and
a third range where the BIS is over 60 and up to 80, and
the determining the target BIS range comprises:
determining a range among the first range, the second range, and the third range that includes the target BIS.

3. The method of claim 2, wherein
when the target BIS is included in the first range,
the evaluating the pain of the target patient comprises:
evaluating the pain of the target patient using the first pain score.

4. The method of claim 2, wherein
when the target BIS is included in the second range,
the evaluating the pain of the target patient comprises:
extracting features for the AU from a facial image of the target patient;
determining the second pain score by inputting the features for the AU into a pre-trained artificial intelligence model; and
evaluating the pain of the target patient using the second pain score.

5. The method of claim 4, wherein
the artificial intelligence model is trained to determine the second pain score based on a training dataset comprising a facial image of a patient and an NRS (numeric rating scale) label corresponding to the pain of the patient.

6. The method of claim 2, wherein
when the target BIS is included in the third range,
the evaluating the pain of the target patient comprises:
evaluating the pain of the target patient using the first pain score and the second pain score.

7. The method of claim 6, wherein
the evaluating the pain of the target patient comprises:
determining a third pain score by calculating a weighted sum of the first pain score and the second pain score, based on a weight determined from the target BIS; and
evaluating the pain of the target patient using the third pain score.

8. The method of claim 1, further comprising:
displaying at least one pain score among the first pain score, the second pain score, and a third pain score, based on monitoring of the target BIS.

9. The method of claim 8, wherein
the displaying the at least one pain score comprises:
providing an alarm to medical staff when at least one of the first pain score, the second pain score, and the third pain score exceeds a preset threshold value.

10. An apparatus for evaluating pain in a pain diagnosis system, the apparatus comprising:
a memory storing a pain evaluation program including one or more instructions; and
a processor that loads the pain evaluation program from the memory and executes the pain evaluation program,
wherein the one or more instructions, when executed by the processor, cause the processor to:
determine a target BIS (bispectral index) range that includes a target BIS measured from a target patient, and
evaluate the pain of the target patient by using at least one of a first pain score related to an ANI (analgesia nociception index) and a second pain score related to an AU (action unit) representing facial expressions, based on the target BIS range.

11. The apparatus of claim 10, wherein
the target BIS range comprises:
a first range where the BIS is 60 or less;
a second range where the BIS is over 80; and
a third range where the BIS is over 60 and up to 80, and
the one or more instructions, when executed by the processor, cause the processor to determine a range among the first range, the second range, and the third range that includes the target BIS.

12. The apparatus of claim 11, wherein
when the target BIS is included in the first range,
the one or more instructions, when executed by the processor, cause the processor to evaluate the pain of the target patient using the first pain score.

13. The apparatus of claim 11, wherein
when the target BIS is included in the second range,
the one or more instructions, when executed by the processor, cause the processor to:
extract features for the AU from a facial image of the target patient,
determine the second pain score by inputting the features for the AU into a pre-trained artificial intelligence model, and
evaluate the pain of the target patient using the second pain score.

14. The apparatus of claim 13, wherein
the artificial intelligence model is trained to determine the second pain score based on a training dataset comprising a facial image of a patient and an NRS (numeric rating scale) label corresponding to the pain of the patient.

15. The apparatus of claim 11, wherein
when the target BIS is included in the third range,
the one or more instructions, when executed by the processor, cause the processor to evaluate the pain of the target patient using the first pain score and the second pain score.

16. The apparatus of claim 15, wherein
the one or more instructions, when executed by the processor, cause the processor to:
determine a third pain score by calculating a weighted sum of the first pain score and the second pain score, based on a weight determined from the target BIS, and
evaluate the pain of the target patient using the third pain score.

17. A pain diagnosis system, comprising:
a BIS measurement unit that measures a target BIS of a target patient;
an ANI measurement unit that measures an ANI of the target patient;
an imaging unit that acquires a facial image of the target patient;
a pain evaluation apparatus that determines a target BIS range that includes the target BIS, and evaluates the pain of the target patient using a pain score determined based on the target BIS range;
a display unit that displays the target BIS, the ANI, and the pain score; and
a monitoring unit that, through monitoring of the target BIS, provides an alarm to medical staff when the pain score exceeds a preset threshold value.

18. A computer-readable recording medium storing a computer program, wherein the computer program, when executed by a processor, causes the processor to:
determine a target BIS (bispectral index) range that includes a target BIS measured from a target patient; and
evaluate the pain of the target patient by using at least one of a first pain score related to an ANI (analgesia nociception index) and a second pain score related to an AU (action unit) representing facial expressions, based on the target BIS range.

19. A computer program stored on a computer-readable recording medium, wherein the computer program, when executed by a processor, causes the processor to:
determine a target BIS (bispectral index) range that includes a target BIS measured from a target patient; and
evaluate the pain of the target patient by using at least one of a first pain score related to an ANI (analgesia nociception index) and a second pain score related to an AU (action unit) representing facial expressions, based on the target BIS range.
